(19) United States
(12) Patent Application Publication
(10) Pub. No.: US 2018/0014830 A1
(43) Pub. Date: Jan. 18, 2018

(54) SYSTEM FOR INSTALLING AN INPLANT, IMPLANT, AND INSTALLATION TOOL

(71) Applicants: UNIVERSITAET DUISBURG-ESSEN, Essen (DE); CORAMAZE TECHNOLOGIES GMBH, München (DE)

(72) Inventors: Till NEUMANN, Essen (DE); Torsten SCHEUERMANN, Munchen (DE); Niklas NATHE, Wolfratshausen (DE)

(21) Appl. No.: 15/546,913

(22) PCT Filed: Apr. 6, 2016
(86) PCT No.: PCT/EP2016/000560

§ 371 Date: Sep. 25, 2017

(30) Foreign Application Priority Data
Apr. 7, 2015 (DE) 10 2015 004 246.7

(51) Int. Cl. A61B 17/12; A61F 2/962; A61F 2/95

(52) U.S. Cl. CPC A61B 17/12109; A61F 2/962; A61B 17/1214; A61B 2017/1205; A61F 2002/9511

(57) ABSTRACT

A system for inserting an implant at an implantation site has a tubular catheter through which the implant to be installed is transported in a longitudinal travel direction to the implantation site. A ring is spaced forward in the direction to the implantation site from the implant and is longitudinally coupled to the implant. A guide wire in the tubular catheter also can pass through the ring. A push tube in the tubular catheter the guide wire is slidable on the guide wire longitudinally forward toward the implantation site and has a front end closer to the implantation site and for which the ring forms an impenetrable stop.

### CROSS REFERENCE TO RELATED APPLICATIONS

This application is the US-national stage of PCT application PCT/EP2016/000560 filed 6 Apr. 2016 and claiming the priority of German patent application 102015004246 itself filed 7 Apr. 2015.

### FIELD OF THE INVENTION

The invention relates to a system for inserting an implant, in particular a self-expanding implant, to an implantation site, preferably to a blood vessel or the heart, comprising a tubular catheter and an implant to be applied that is inserted therein, which can be transported through the tubular catheter to the implantation site.

### BACKGROUND OF THE INVENTION

Moreover, the invention also relates to an insertion device for inserting an implant to an implantation site of a body with a tubular catheter for forming a chamber from a location outside a body to an implantation site inside of the body, as well as to an implant for use with such an insertion device and/or a system of the above-described type.

It is generally known in the prior art, for example in operations on the human or also animal body, to position implants within the body at an implantation site. The implant that is then present at the implantation site is frequently intended to take over a medically necessary function.

Some examples of typical insertions are stents as implants that are used in blood vessels in order to reopen an occluded blood vessel or prevent imminent occlusion. Another noteworthy insertion consists in implanting a prosthetic heart valve in a heart or also positioning a closure element as an implant in the passage region of the heart valves while maintaining the natural heart valves, a procedure that remedies so-called heart valve insufficiency of the mitral valve or of the tricuspid valve, for example.

The invention that will be described in the following is especially preferably intended for use in the above-described operations but is not limited thereto in principle.

The insertion of implants, particularly implants to be implanted in a vessel or the heart, is frequently performed in such a way that a travel path is opened up by a tubular catheter through which the implant to be applied can be slid from a location outside the body to the implantation site.

It is possible to insert both implants that retain their original size and those that are self-expanding or are actively expanded at the implantation site, for example by a balloon.

According to the known methods, the procedure followed is usually one in which the implant to be applied, after previous positioning of the tubular catheter with its open rear end outside the body, is inserted into the catheter and then advanced by a pushing device, a guide wire, for example, through the tubular catheter to the implantation site, after which it is positioned at that location and optionally expanded either actively or automatically.

In this type of procedure, it is regarded as problematic that the entire implant to be installed is in the direction of displacement at a front end of the pushing device that is exerting the pushing force and therefore, as a result of the friction between the implant and the inner walls of the tubular catheter, the implant tends to be enlarged in cross section, which renders further advance difficult. Implantations of this type must therefore be carried out with great care in order to reduce this disadvantageous effect.

This disadvantageous effect is significant especially when self-expanding implants are to be applied in this way, since they are already predisposed to expanding on their own without the effect of a displacing force and thus tend to generate increased friction on the inner walls of the tubular catheter. This effect is problematic especially in the case of long and curvy travel paths.

### OBJECT OF THE INVENTION

It is an object of the invention to provide a system of the type described above, an insertion device of the type described above, and an implant suitable for same with which the above-mentioned disadvantageous effect during the sliding insertion of an implant through the tubular catheter is eliminated.

### SUMMARY OF THE INVENTION

This object is achieved in a system where the implant of such a system has an annulus through which a guide wire lying in the tubular catheter is placed, and a inner catheter is provided in the tubular catheter that surrounds the guide wire and can be slid on the guide wire toward the implantation site and for whose front end closer to the implantation site the annulus forms an impenetrable stop.

A passage can be formed, for example, by a simple structure such as a ring or sleeve, particularly by one whose inside diameter is greater than the diameter of the guide wire and smaller than the outside diameter of the inner catheter.

An insertion device according to the invention has, in addition to a tubular catheter designed to form a chamber and therefore also referred to herein as a tubular catheter, both a guide wire that can be inserted into the first catheter and displaced therein to an implantation side and, in addition, an inner catheter that can be inserted into the tubular catheter and can be pushed over the guide wire and displaced along the guide wire in the tubular catheter for the purpose of moving an implant. In terms of the invention, the inner catheter that is inserted for the purpose of moving the implant, is also referred to as a push tube.

An implant according to the invention that is provided for use with an above-mentioned system and/or an above-mentioned insertion device, is modified relative to previous implants in that is has an annulus, for example in the form of a ring/sleeve, that has an inner passage through which a guide wide can be passed, with the inner passage dimensioned such that the ring for the open end of a tubular catheter guided on the guide wire, namely, in particular, of the second catheter or push tube of the above-mentioned insertion device or system, forms a stop.

The term “guide wire” does not necessarily mean that it is a wire made of metal, although that can be the case.

An essential core idea of the invention is that, unlike in the prior art, when the implant is moved using the system according to the invention or the insertion device according to the invention, pushing force no longer acts on the implant as a whole on the rear side of the implant turned away from the implantation site; rather, due to the fact that the ring of the implant is on the front side of the implant closer to the implantation site, the pushing force of the push tube acting on the ring pulls along the remaining portion of the implant that is attached to the ring.

In this way, the implant is pulled longitudinally when moved in the catheter and not compressed as in the prior art. Frictional forces between the implant and the inner wall of the tubular catheter are thus reduced by this method of insertion. Self-expanding implants can therefore also be especially preferably applied with this system or insertion device, since the pulling force exerted by the ring on the rest of the implant at least partially counteracts the self-expansion force.

In order to prevent the feed force acting from the open end of the push tube located in the tubular catheter on the ring from having any other effect at all, or at least no substantial effect on the rest of the implant, in a first aspect of the invention the implant is provided in the tubular catheter laterally next to the guide wire and beside the push tube and the guide wire penetrates only through the ring of the implant but not through the rest of the implant.

According to a second aspect of the invention, however, the guide wire and by the push tube pass longitudinally completely or at least partially through the implant in the tubular catheter in the direction toward the implantation site. Such an embodiment can be provided particularly if an implant is to be applied that has an inner passage, optionally even in its compressed state, that is large enough to pass the guide wire and the push tube through. This can be the case particularly in stents that are to be implanted.

In principle, any implant that is known from the prior art can be used as an implant of the type according to the invention, provided that it has the inventive ring on the side closer to the implantation site through which the guide wire can be pushed and that forms the inventive stop for the push tube that is guided over the guide wire. For instance, the above-mentioned stents or also artificial heart valves or occlusive bodies for reducing or eliminating a heart valve insufficiency can also be provided with such a ring according to the invention, a ring or a sleeve in the simplest of cases.

According to the invention the arrangement of a ring on one of the above-described implants, or even on any other implant known thus far or to be developed in the art, is performed exclusively for the purpose of transportation through the tubular catheter, and that the ring as well as, optionally, elements provided for the attachment thereof to the implant, no longer performs any function after the insertion of the implant.

According to another aspect of the invention, however, the ring of the implant is part of an anchor element of the implant with which the implant can be installed at an implantation site after implantation at the implantation site, particularly after the expansion of at least the anchor element, particularly also of the rest of the implant in the body.

In such an embodiment, the annulus (for example ring or sleeve) thus assumes not only a mere transport function but can also take be, at least in part, a functional component in the fastening of the implant in a body lumen such as a vessel or heart ventricle, for example.

In the two possible embodiments cited above, according to the invention the ring of the implant is spaced from the rest of the implant by at least two preferably elastically resilient strips. Their spacing is created here relative to the position that the implant occupies during the transport thereof through the tubular catheter, such that the preferably elastically resilient strips extend forward from the front end of the implant toward the implantation site and toward the ring.

Especially if these strips and, optionally, the ring fastened thereto, constitute a part of a anchor element of the implant, according to the invention the strips can be moved against a restoring force into a first, particularly compressed position in which the strips assume a position in which they are close to each other.

This first position is reached by having the pushing force exerted on the ring by the push tube that is guided on the guide wire, such that the ring pulls the rest of the implant behind it during transport through the tubular catheter.

This first particularly compressed position is already present for the purpose of fitting the implant into the tubular catheter, for which purpose the implant is enclosed by a sheath by the manufacturer, for example, that holds the implant against a restoring force in the first position, so that, for the purpose of transport, the implant is already inserted in this existing first position into the open end of the tubular catheter located outside the body, and the outer sheath around the implant is removed, for example as a result of the tearing-open of the sheath wall.

In this position, the strips that extend between the ring and the rest of the implant are close to each other and, insofar as the implant is located together with the guide wire in the tubular catheter, close to the guide wire as well, particularly at a distance to one another that is shorter than the inner cross-sectional dimension of the tubular catheter.

In this position, in which the strips are positioned near to one another, the ring of the implant is still spaced forward of the rest of the implant.

Moreover, according to the invention there is also a second, particularly expanded position in which the implant can be moved, particularly by a restoring force, with the strips in a spread position relative to one another in this second position, and with the ring close position to the rest of the implant.

Where it is stated herein that the strips can be in a close and a spread position relative to one another, this means that the strips are closer to each other in the closed position than in the spread position. Accordingly, the denotations “close” and “spaced” are to be understood as being relative and not absolute. The same applies to the same information relative to the longitudinal position of the ring relative to the rest of the implant.

Through the provision of these two possible positions that the implant can assume, with the second position being brought about from the first position as a result of the restoring force, a self-expanding effect can be achieved as a result of which the strips move away from each other during the expansion and have a greater distance between them, so that an anchor element of the implant can be formed by the strips themselves that is used to fix the implant in place in a body lumen, for example a vessel or a heart chamber, after implantation thereof.

For this purpose, the strips can be embodied, through construction from a shape-memory alloy, for example, such that they rest against the inner wall of the body lumen in a contacting manner, at least in a portion of the extension thereof.

In one embodiment, the restoring force can also be achieved by using a shape-memory alloy (such as nitinol, for example) as the material at least for the strips, and the restoring force is only generated upon heating of the strips to body temperature.

A provision can also be made according to the invention that the ring of the implant is guided between the two previously mentioned positions in one direction, or moves in a direction that corresponds to the direction of extension of the guide wire, so that it is especially ensured in this way that, when the implant moves inside the tubular catheter as a result of the pushing force of the push tube acting on the ring, the ring is pushed toward the spaced position and, precisely as a result of this, a force that counteracts the self-expansion is produced in the implant or anchor element, as already described above.

In a preferred use, the implant can constitute a device for eliminating or reducing heart-valve insufficiency. In an embodiment that is preferred according to the invention, such an implant comprises an occluding body that can be provided between heart valves and an annulus, preferably a ring, that is attached to the occluding body by preferably expandable strips, with the strips forming an anchor element particularly in the expanded state in order to fix the occluding body in place in the heart after positioning, as a result of the preferably expanded strips resting against the wall of the heart.

### BRIEF DESCRIPTION OF THE DRAWING

Embodiments of the invention are described in further detail with reference to the following figures in which:

FIG. 1 is a partly schematic side sectional view of the invention;

FIG. 2 shows the implant partly pushed out of the implantation catheter; and

FIG. 3 is an end view of a detail of the structure.

### SPECIFIC DESCRIPTION OF THE INVENTION

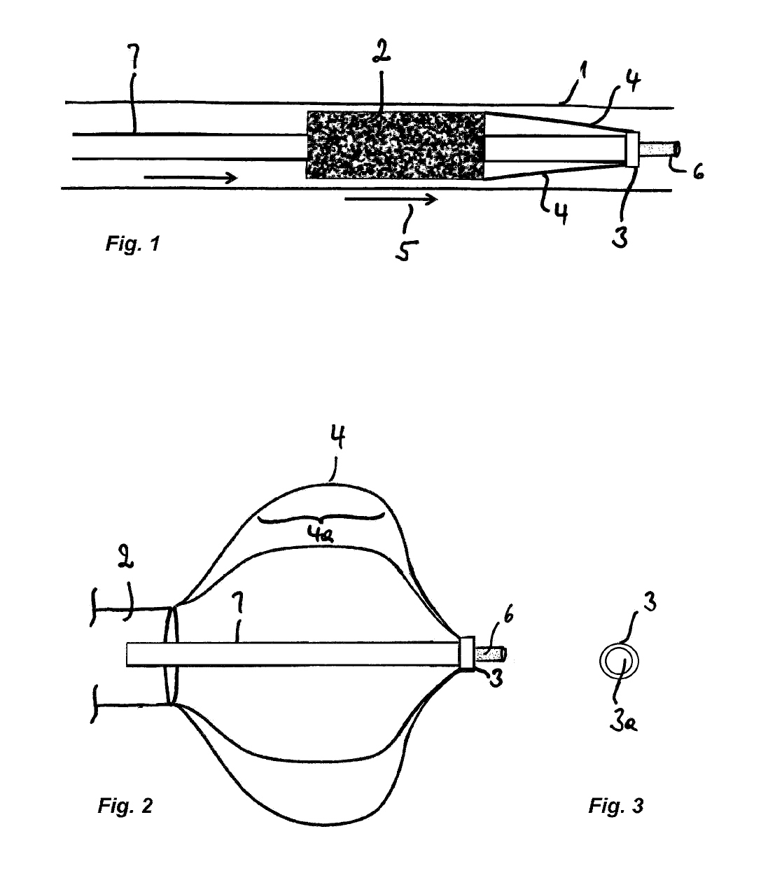

FIG. 1 is a first partial section through a system according to the invention with a first tubular, tubular catheter 1 that, in order to open up a travel path from a location outside a human or animal body for example, is moved to a desired implantation site.

An implant 2 according to the invention is held inside the tubular catheter 1 and has an inventive ring 3 that is connected by several strips 4 to the rest of the implant and, relative to a transport direction 5, is located on the end of the implant 2 closer to the implantation site.

The ring 3 has an inner passage 3a through which passes a guide wire 6 that extends coaxially through the tubular catheter 1 and that extends in the tubular catheter to the implantation site.

The implantation can be performed in that the tubular catheter 1 is first slid into place, after which the guide wire 6 is inserted into it, and then the ring 3 of the implant is fitted over the free end of the guide wire 6 that is outside the body.

After this, as shown in FIG. 1 for the system shown therein, a second tubular push tube 7 can be pushed onto the guide wire 6, so that the free/open front end thereof that is closer to the implantation site is guided on the guide wire 6 toward the ring 3 of the implant 2, with the ring 3 forming a stop for the open end of the push tube 7, which open end of the push tube 7 cannot penetrate through the ring 3.

Accordingly, if the push tube 7 is pushed forward by a surgeon to the implantation site, for example, pressure is exerted on the ring 3 of the implant in the direction of the arrow 5, and the ring 3 is pushed along toward the implantation site and pulls along behind it the rest of the implant 2 connected via the strips 4.

In this way, the implant 2 can be transported to the intended implantation site, released from the tubular catheter 1 and, depending on its design, either remain in its shape, be actively expanded, or optionally expand automatically.

FIG. 2 shows an embodiment in which the strips 4 form a self-expanding anchor element together with the ring 3, for which purpose the strips 4 can be made of a shape-memory alloy such as nitinol (nickel-titanium alloys) or certain polymers or of another resilient material.

FIG. 2 shows the implant 2 at the implantation site in a state in which the implant 2 is pushed forward out of the outer tubular catheter 1 and no pushing force is exerted by the push tube 7 on the ring 3. This can be achieved by retracting the push tube 7, for example.

Due to the resilient elasticity of the strips 4 and the resulting force that brings about a self-expansion, the spacing between the individual strips 4 relative to each other increases, and the ring 3 is pulled back toward the rest of the implant 2.

This results in a bulging of the strips 4 in the region between the ring and the rest of the implant, that is, the strips assume a position in which they are farther from one another. Overall, the spacing of the strips 4 relative to one another results in a shape of the arrangement of the strips 4 and the ring 3 in which the strips are convexly bulged in a direction perpendicular to the guide wire 6, i.e. radially outward therefrom.

The convexly structured region 4a makes the strips engage against inner walls of a body lumen, for example a heart chamber.

FIG. 2 clearly shows overall that the arrangement of the strips 4 and the ring 3 can form a cage-like anchor element that adapts automatically to the inner dimensions of a body lumen, thus contributing to a fastening of the implant in the body lumen.

In one exemplary insertion, the rest of the implant 2 that is provided on what are the rear ends of the strips here, can be embodied, for example, as an occlusive body that is provided in order to eliminate or at least to reduce heart-valve insufficiency.

1. A system for inserting an implant at an implantation site, the system comprising:
a tubular catheter through which the implant to be installed is transported in a longitudinal travel direction to the implantation site,
a ring spaced forward in the direction to the implantation site from the implant and longitudinally coupled th the implant;
a guide wire in the tubular catheter; and
a push tube in the tubular catheter surrounding the guide wire and slidable on the guide wire longitudinally forward toward the implantation site and having a front end closer to the implantation site and for which the ring forms an impenetrable stop.

2. The system defined in claim 1, wherein the implant is provided inside the tubular catheter laterally adjacent the guide wire and push tube and that the guide wire extends only through the ring of the implant.

3. The system defined in claim 1, wherein the guide wire and the push tube extend completely or partly through the implant in the tubular catheter in the longitudinal direction toward the implantation site.

4. The system defined in claim 1, wherein the ring of the implant is part of a anchor element of the implant with which the implant can be secured in the implantation site after implantation.

5. The system defined in claim 1, wherein the ring of the implant is spaced apart from the rest of the implant by at least two elastically resilient strips.

6. The system defined in claim 5, wherein the strips can be moved against a restoring force into a first, particularly compressed position, in which the strips assume a position in which they are close to each other and the ring is spaced forward from the rest of the implant, and moved by the restoring force into a second expanded position, in which the strips are outwardly spread relative to one another and the ring is longitudinally close to the rest of the implant.

7. The system defined in claim 6, wherein the ring of the implant is guided between the first and second positions along a longitudinal direction generally parallel to a direction of extension of the guide wire.

8. The system defined in claim 1, wherein the ring and the push tube have interfitting coupling formations that are detachably fastened to one another in a nonpositive manner.

9. An insertion device for inserting an implant to an implantation site of a body, the device comprising:
a tubular catheter forming a passage extending in a longitudinal direction from a location outside a body to an implantation site inside the body,
a guide wire that can be pushed longitudinally into the catheter and displaced therein to the implantation site; and
a push tube that can be pushed into the tubular catheter and pushed over the guide wire and displaced along the guide wire in the tubular catheter for moving the implant to the implantation site.

10. An implant for use with an insertion device defined in claim 9, wherein it has a ring on its side closer to the implantation site that has an inner passage through which a guide wire can be passed, and that the passage is dimensioned such that the ring forms a stop for the open end of a push tube guided on the guide wire.

11. A system for inserting an implant in a longitudinally forward direction into an implantation site inside a body, the system comprising:
a tubular catheter having a front end adapted to inserted through a body vessel or duct to the implantation site while a rear end of the catheter remains outside the body, the implant being slidable forward from the rear end outside the body through the catheter to the implantation site inside the body;
a guide wire passing through the catheter from the rear end thereof and past the implant;
a ring longitudinally forward of the implant and forming a passage through which passes the guide wire;
formations longitudinally coupling the ring to the catheter; and
a push tube coaxially surrounding the guide wire, coaxially surrounded by the catheter, and having a front end longitudinally abuttable against the ring and of too great a diameter to pass longitudinally through the ring, whereby the push tube can slide the ring and implant forward out of the catheter.

12. The implant installing system defined in claim 11, wherein the push tube and guide wire pass coaxially through the implant.

13. The implant installing system defined in claim 11, wherein the formations are formed by a plurality of strips extending longitudinally between the ring and the implant and constructed so as to arc radially outward when not radially compressed such that when pushed out of the catheter by the push tube the strips spread.

14. The implant installing system defined in claim 11, wherein the implant is formed with a longitudinally throughgoing hole through which passes the push tube surrounding the wire, the ring forming a passage of a diameter greater than a diameter the guide wire but smaller than a diameter of the push tube so that the guide wire can pass freely through the ring but the push tube cannot.
